(19) United States
(12) Patent Application Publication
(10) Pub. No.: US 2010/0113530 A1
(43) Pub. Date: May 6, 2010

(54) S1P LYASE INHIBITORS FOR THE TREATMENT OF CEREBRAL MALARIA

(76) Inventors: Philip Manton Brown, The Woodlands, TX (US); Constance Ann Marjory Finney, Toronto (CA); Kevin Charles Kain, Toronto (CA); Tamas Oravecz, The Woodlands, TX (US); Stephen Chris Pappas, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 12/608,426

(22) Filed: Oct. 29, 2009

Related U.S. Application Data
(60) Provisional application No. 61/109,982, filed on Oct. 31, 2008.
(60) Provisional application No. 61/109,987, filed on Oct. 31, 2008.

(51) Int. Cl. A61K 31/4439; A61K 31/427; A61K 31/422; A61K 31/4196; A61K 31/4164; A61K 31/4188

(52) U.S. Cl. 514/341; 514/365; 514/378; 514/383; 514/406

(57) ABSTRACT

Methods and compositions for treating, managing, and/or preventing cerebral malaria are disclosed.

This application claims priority to U.S. provisional application Nos. 61/109,982 and 61/109,987, both filed Oct. 31, 2008, the entireties of which are incorporated herein by reference.

### FIELD OF THE INVENTION

This application is directed to methods of treating, managing, and/or preventing cerebral malaria, and compositions useful therein.

### BACKGROUND

### 2.1. Cerebral Malaria

More than two million people, most of whom are African children, die each year of malaria. Golenser, J., et al., Int. J. Parasitology 36:583-593, 583 (2006). Eradication of the disease “has been hampered by the development of Plasmodium (especially Plasmodium falciparum, the most abundant and dangerous causative species) resistant to currently available anti-malarial drugs.” Id.

One of the most severe complications of P. falciparum infection is cerebral malaria (CM), which is expressed in about 7 percent of P. falciparum malaria cases. CM manifests as coma (Blantyre coma scale ≦2 or Glasgow coma scale ≦8), P. falciparum on blood smear, and no other known cause for coma. John, C. C., et al., Pediatrics 122:e92-e99 (2008). CM affects an estimated 785,000 children in sub-Saharan Africa every year, with an average mortality rate of 18.6 percent. Golenser at 586; John at e93. A recent study found that one in four children who survive CM suffer long-term cognitive impairment. John, id.

Although the pathogenesis of CM is unclear, a simplified explanation is that the adherence “to endothelial cells and the sequestration of parasitized erythrocytes and immune cells in brain capillaries cause an inflammatory process and the release of other neurotoxic molecules.” Golenser at 584. It is possible to treat some CM cases with anti-malaria drugs. Id. at 586. But there is an “irreversible stage after which the patient dies, despite massive anti-parasitic treatment.” Id. Thus, a number of adjunctive treatments have been suggested, some of which have shown promise, but many of which have not. See, id. at 586-591.

### 2.2 SIP Pathway

Sphingosine-1-phosphate (SIP) is a bioactive molecule with potent effects on multiple organ systems. Saba, J. D. and Hla, T. Circ. Res. 94:724-734 (2004). The compound binds with low affinity to five related G-protein coupled receptors, S1P1-5, formerly termed endothelial differentiation gene (EDG) receptor-1, -5, -3, -6, and -8, respectively. Brinkmann, V., Pharmacol. & Therapeutics 115:84-105, 85 (2007). The receptor subtypes S1P1, S1P2, and S1P3 are widely expressed in the cardiovascular system. Id. at 85-86. S1P1 is the dominant receptor on lymphocytes, and regulates their egress from secondary lymphatic organs. Id.

Numerous agonists of the SIP receptors have been reported and proposed as potential therapies in diseases that include host-versus-graft disease, rheumatoid arthritis and multiple sclerosis (MS). The S1P1 agonist FTY720 (fingolimod) in particular has been extensively studied, and is currently in clinical trials for the treatment of MS. Id. at 95-100.

It appears possible to treat some diseases by affecting other parts of the SIP pathway, as well. For example, an inhibitor of the enzyme S1P lyase, which catalyzes the cleavage of S1P into ethanolamine phosphate and a long-chain aldehyde, is effective in rheumatoid arthritis models, and is currently in clinical trials. Oravecz, T. et al., “Sphingosine-1-Phosphate Lyase is a Potential Therapeutic Target in Autoimmune Diseases Including Rheumatoid Arthritis,” Presentation 1833, American College of Rheumatology Scientific Meeting (San Francisco, Oct. 28, 2008); Pappas, C., et al., “LX2931: A Potential Small Molecule Treatment for Autoimmune Disorders,” Presentation 351, American College of Rheumatology Scientific Meeting (San Francisco, Oct. 26, 2008). See also U.S. patent application publication no. 2007/0208063; U.S. patent application Ser. No. 12/038,872.

### SUMMARY OF THE INVENTION

This invention encompasses methods treating, managing, and/or preventing cerebral malaria, which comprise administering to a patient in need thereof a therapeutically or prophylactically effective amount of an SIP lyase inhibitor. Particular SIP lyase inhibitors are compounds of the formula:

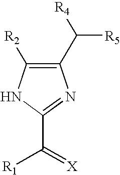

and pharmaceutically acceptable salts thereof, wherein: X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; R₅ is N(R_{5A})₂, hydrogen, hydroxy, or optionally substituted alkyl, aryl, alkylaryl, arylalkyl, heteroalkyl, heterocycle, alkylheterocycle, or heterocyclealkyl; and each of R_{1A}, R_{2A}, R_{3A}, R_{4A}, and R_{5A} is independently hydrogen or optionally substituted alkyl, aryl, alkylaryl, arylalkyl, heteroalkyl, heterocycle, alkylheterocycle, or heterocyclealkyl.

In some methods, the S1P lyase inhibitor is administered adjunctively with one or more additional active agents.

This invention also encompasses pharmaceutical compositions useful in the treatment, management, and/or prevention of CM.

### BRIEF DESCRIPTION OF THE FIGURES

Certain aspects of this invention can be understood with reference to the following figures:

FIG. 1 shows the effect of an S1P lyase inhibitor on the lymphocytes of mice in the cerebral malaria model described below in the Examples.

FIG. 2 shows the effect of an S1P lyase inhibitor administered i.p. and gavage on the survival of mice in the cerebral malaria model described below in the Examples.

### DETAILED DESCRIPTION

This invention is directed to the use of S1P receptor agonists for the treatment, management and/or prevention of cerebral malaria (CM). The invention is based, in part, on Applicants' discovery that CM may be treated by modulating the SIP pathway. For example, Applicants have discovered that both agonizing the SIP receptor and inhibiting SIP lyase can provide protection against CM in the well-established murine model of the disease. See, e.g., U.S. provisional application No. 61/109,991, filed Oct. 31, 2008, U.S. provisional application 61/229,970, filed Jul. 30, 2009, and U.S. provisional application No. 61/109,982, filed Oct. 31, 2009.

### 5.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include —OCH₃, —OCH₂CH₃, —O(CH₂)₂CH₃, —O(CH₂)₃CH₃, —O(CH₂)₄CH₃, —O(cyclopenyl) and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Particular heterocycles are 5- to 13-membered heterocycles containing 1 to 4 heteroatoms selected from nitrogen, oxygen, and sulphur. Others are 5- to 10-membered heterocycles containing 1 to 4 heteroatoms selected from nitrogen, oxygen, and sulphur. Examples of heterocycles include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “stereoisomeric mixture” encompasses racemic mixtures as well as stereomerically enriched mixtures (e.g., R/S=30/70, 35/65, 40/60, 45/55, 55/45, 60/40, 65/35 and 70/30).

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 5.2. S1P Lyase Inhibitors

Embodiments of this invention employ compounds of formulae I.A and II.A, described below. Compounds of both formulae have been shown to inhibit S1P lyase. See, e.g., U.S. patent application publication no. US-2007-0208063-A1 and U.S. Pat. No. 7,598,280.

Particular compounds are of formula I.A:

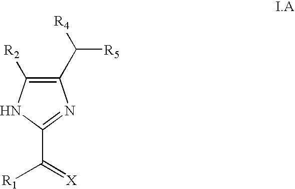

and pharmaceutically acceptable salts thereof, wherein: X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; R₅ is N(R_{5A})₂, hydrogen, hydroxy, or optionally substituted alkyl, aryl, alkylaryl, arylalkyl, heteroalkyl, heterocycle, alkylheterocycle, or heterocyclealkyl; and each of R_{1A}, R_{2A}, R_{3A}, R_{4A}, and R_{5A} is independently hydrogen or optionally substituted alkyl, aryl, alkylaryl, arylalkyl, heteroalkyl, heterocycle, alkylheterocycle, or heterocyclealkyl.

Particular compounds of formula I.A are such that if X is O; R₁ is alkyl of 1 to 4 carbons, phenyl, benzyl or phenylethyl; R₂ is hydrogen; and one of R₄ and R₅ is hydroxyl; the other of R₄ and R₅ is not alkyl of 1 to 6 carbons, hydroxyalkyl of 1 to 6 carbons, polyhydroxyalkyl of 1 to 6 carbons having up to one hydroxyl per carbon, polyacetylalkyl of 1 to 6 carbons having up to one acetyl per carbon, phenyl, benzyl or phenylethyl.

In particular embodiments, the compound is not 2-acetyl-4-tetrahydroxybutylimidazole, 1-(4-(1,1,2,2,4-pentahydroxybutyl)-1H-imidazol-2-yl)ethanone, 1-(2-acetyl-1H-imidazol-4-yl)butane-1,1,2,2-tetrayl tetraacetate, or 1-(2-acetyl-1H-imidazol-4-yl)butane-1,1,2,2,4-pentayl pentaacetate.

A particular embodiment employs compounds of formula I.B:

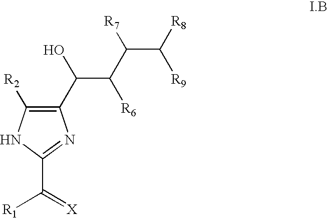

and pharmaceutically acceptable salts thereof, wherein: X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is OR_{8A}, OC(O)R_{8A}, N(R_{8B})₂, NHC(O)R_{8B}, hydrogen, or halogen; R₉ is CH₂OR_{9A}, CH₂OC(O)R_{9A}, N(R_{9B})₂, NHC(O)R_{9B}, hydrogen, or halogen; each of R_{1A}, R_{2A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl; and each of R_{6B}, R_{7B}, R_{8B} and R_{9B} is independently hydrogen or alkyl optionally substituted with one or more hydroxy or halogen groups; Particular compounds of formula I.B are such that: 1) if X is O, R₁ is alkyl of 1 to 4 carbons, phenyl, benzyl or phenylethyl, and R₂ is hydrogen, at least two of R₆, R₇, R₈ and R₉ are not hydroxyl or acetate; 2) if X is O, R₁ is methyl, R₂ is hydrogen, R₆ and R₇ are both hydroxyl, and one of R₈ and R₉ is hydrogen, the other is not NHC(O)R_{9B}; 3) if X is O, R₁ is OR_{1A}, R_{1A} is hydrogen or lower alkyl, and R₂ is hydrogen, at least one, but not all, of R₆, R₇, R₈ and R₉ is hydroxyl or acetate.

Particular compounds of the invention are of formula I.B(a):

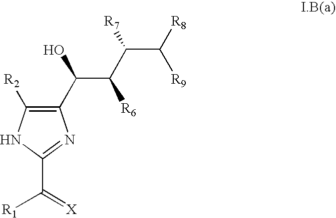

Others are of formula I.C:

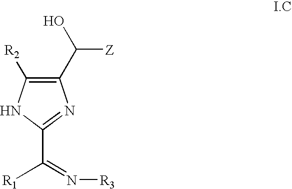

wherein: Z is optionally substituted alkyl; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; and each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

Another embodiment of the invention employs compounds of formula I.D:

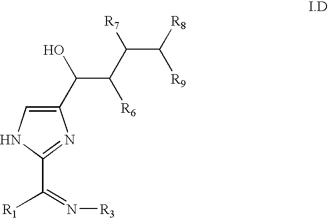

and pharmaceutically acceptable salts thereof, wherein: R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is OR_{8A}, OC(O)R_{8A}, N(R_{8B})₂, NHC(O)R_{8B}, hydrogen, or halogen; R₉ is CH₂OR_{9A}, CH₂OC(O)R_{9A}, N(R_{9B})₂, NHC(O)R_{9B}, hydrogen, or halogen; and each of R_{1A}, R_{3A}, R_{6A}, R_{5A}, R_{8A} and R_{9A} is independently hydrogen or optionally substituted alkyl, aryl, alkylaryl, arylalkyl, heteroalkyl, heterocycle, alkylheterocycle, or heterocyclealkyl.

Particular compounds are of formula I.D(a):

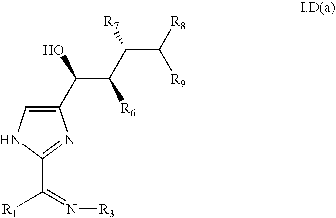

With regard to each of the formulae shown above that contain the moieties described below, certain embodiments of the invention are such that:

In some, X is O. In others, X is NR₃.

In some, R₁ is hydrogen. In others, R₁ is optionally substituted lower alkyl. In others, R₁ is NHOH. In others, R₁ is OR_{1A} and R_{1A} is, for example, hydrogen or optionally substituted lower alkyl.

In some, R₂ is hydrogen. In others, R₂ is not hydrogen. In others, R₂ is nitrile. In others, R₂ is optionally substituted lower alkyl. In others, R₂ is OR_{2A}. In others, R₂ is C(O)OR_{2A}. In some, R_{2A} is hydrogen or optionally substituted lower alkyl.

In some, R₃ is OR_{3A}. In others, R₃ is N(R_{3A})₂ or NHC(O)R_{3A}. In others, R₃ is NHSO₂R_{3A}. In some, R_{3A} is hydrogen or optionally substituted lower alkyl. In others, R_{3A} is optionally substituted aryl or heterocycle.

In some, R₄ is OR_{4A}. In others, R₄ is halogen.

In some, R₅ is N(R_{5A})₂. In others, R₅ is hydrogen. In others, R₅ is hydroxyl. In others, R₅ is heteroalkyl (e.g., alkoxy). In others, R₅ is optionally substituted alkyl. In others, R₅ is optionally substituted aryl.

In some, one or more of R₆, R₇, R₈, and R₉ is hydroxy or halogen. In some, all of R₆, R₇, R₈, and R₉ are hydroxyl or acetate.

In some, Z is alkyl optionally substituted with one or more hydroxyl, acetate or halogen moieties.

This invention also employs compounds of formula II:

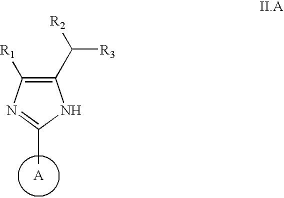

and pharmaceutically acceptable salts and thereof, wherein: A is an optionally substituted heterocycle; R₁ is OR_{1A}, OC(O)R_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; and each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

Particular compounds are of formula II.A (a) or II.A (b):

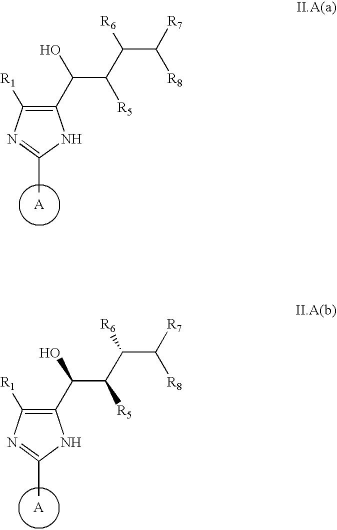

wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

One embodiment of the invention employs compounds of formula II.B:

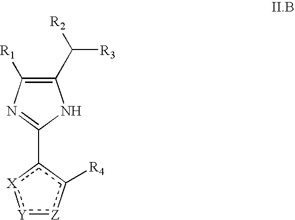

and pharmaceutically acceptable salts thereof, wherein: X is CR₄, CHR₄, N, NR₉, O or S; Y is CR₄, CHR₄, N, NR₉, O or S; Z is CR₄, CHR₄, N, NR₉, O or S; R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl; each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; each R₉ is independently hydrogen or optionally substituted alkyl, aryl, alkylaryl, arylalkyl, heteroalkyl, heterocycle, alkylheterocycle, or heterocyclealkyl; and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

Particular compounds are of formulae II.B(a) or II.B(b):

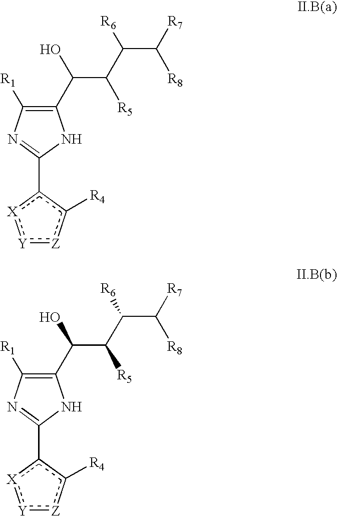

wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

Another embodiment encompasses compounds of formula II.C:

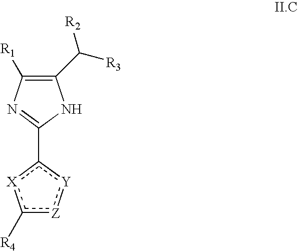

and pharmaceutically acceptable salts thereof, wherein: X is CR₄, CHR₄, N, NR₉, O or S; Y is CR₄, CHR₄, N, NR₉, O or S; Z is CR₄, CHR₄, N, NR₉, O or S; R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl; each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; each R₉ is independently hydrogen or optionally substituted alkyl, aryl, alkylaryl, arylalkyl, heteroalkyl, heterocycle, alkylheterocycle, or heterocyclealkyl; and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

Particular compounds are of formulae II.C(a) or II.C(b):

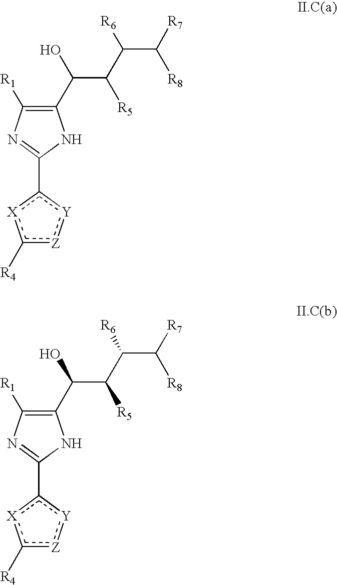

wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

Referring to the various formulae disclosed above (e.g., formulae II.A, II.B and II.C), as applicable, in some compounds of the invention, A is a 5-membered optionally substituted heterocycle. Examples include optionally substituted dihydro-imidazole, dihydro-isoxazole, dihydro-pyrazole, dihydro-thiazole, dioxolane, dithiolane, dithiole, imidazole, isoxazole, isoxazolidine, oxathiolane, and pyrazole. In one embodiment, A is not optionally substituted furan, thiophene or pyrrole.

In some compounds, A is a 6-membered optionally substituted heterocycle (e.g., pyrimidine).

In some, X is CR₄ or CHR₄. In some, X is N or NR₉. In some, X is O or S.

In some, Y is CR₄ or CHR₄. In some, Y is N or NR₉. In some, Y is O or S.

In some, Z is CR₄ or CHR₄. In some, Z is N or NR₉. In some, Z is O or S.

In some, X is N and Y is O. In some, X is N and Y is NR₉. In some, X is N and Y is S. In some, X is N and Z is O. In some, X is N and Z is NR₉. In some, X is N and Z is S. In some, X is N, Y is N, and Z is NR₉.

In some, R₁ is hydrogen. In some, R₁ is nitrile. In some, R₁ is optionally substituted lower alkyl. In some, R₁ is OR_{1A} or C(O)OR_{1A} and R_{1A} is, for example, hydrogen or optionally substituted lower alkyl.

In some, R₂ is OR_{2A}. In some, R₂ is OC(O)R_{2A} and R_{2A} is, for example, hydrogen. In some, R₂ is halogen.

In some, R₃ is optionally substituted alkyl (e.g., alkyl substituted with one or more halogen or OR_{3A} moieties, wherein R_{3A} is, for example, hydrogen or acetate). In some, R₃ is hydrogen. In some, R₃ is hydroxyl. In some, R₃ is optionally substituted heteroalkyl (e.g., alkoxy). In some, R₃ is heteroalkyl substituted with one or more halogen, hydroxyl or acetate.

In some, R₄ is hydrogen or optionally substituted alkyl, aryl or alkylaryl.

In some, each of R₅, R₆, R₇, and R₈ is hydrogen or halogen. In some, one or more of R₅, R₆, R₇, and R₈ is hydroxyl or acetate. In some, all of R₅, R₆, R₇, and R₈ are hydroxyl.

In some, R₉ is hydrogen or optionally substituted alkyl, aryl or alkylaryl.

Compounds of the invention may contain one or more stereocenters, and can exist as racemic mixtures of enantiomers or mixtures of diastereomers. This invention encompasses stereomerically pure forms of such compounds, as well as mixtures of those forms. Stereoisomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N.Y., 1962); and Wilen, S. H., Tables of Resolving Agents and Optical Resolutions, p. 268 (E. L. Eliel, Ed., Univ. of Notre Dame Press, Notre Dame, Ind., 1972).

This invention further encompasses stereoisomeric mixtures of compounds disclosed herein. It also encompasses configurational isomers of compounds disclosed herein, either in admixture or in pure or substantially pure form, such as cis (Z) and trans (E) alkene isomers and syn and anti oxime isomers.

Compounds of the invention can be prepared by methods known in the art, including those disclosed in U.S. patent application publication no. US-2007-0208063-A1 and U.S. Pat. No. 7,598,280.

### 5.3. Additional Active Agents

Some embodiments of the invention employ one or more active agents in addition to an S1P lyase inhibitor. Examples of such additional agents include anti-malarial drugs (e.g., quinine, quinidine, and artemisinin derivatives such as artemether and artesunate), osmotic diuretics (e.g., mannitol and urea), anti-convulsants (e.g., diazepam, phenyloin, phenobarbital, and phenobarbitone), anti-pyretics (e.g., paracetamol), anti-oxidants, and anti-inflammatory drugs (e.g., NSAIDS, steroids, cyclosporin, thalidomide, revlimid, anti-TNF antibodies (e.g., infliximab, etanercept), and pentoxifylline). Others include curdlan sulfate, curcumin, and LMP-420.

### 5.4. Methods of Use

This invention encompasses methods of preventing, managing and treating CM, which comprise administering to a patient a therapeutically or prophylactically effective amount of an S1P lyase inhibitor. The amount of drug, dosing schedule, and route of administration will vary depending on the drug and the patient, and can readily be determined by those of ordinary skill in the art. Because oral administration of drugs may be difficult in some CM patients, preferred routes of administration include i.v. and i.m.

In some embodiments of the invention, the S1P lyase inhibitor is administered adjunctively with one or more additional active agents. Administration of the two or more drugs may be concurrent (e.g., in the same dosage form, or in separate dosage forms administered to the patient at approximately the same time), but need not be.

Methods of treating and managing CM are suitable for patients exhibiting one or more symptoms of CM, including coma (Blantyre coma scale ≦2 or Glasgow coma scale ≦8), P. falciparum on blood smear, and no other known cause for coma. Methods of preventing CM are suitable for patients at risk of CM, e.g., patients having P. falciparum on blood smear and optionally exhibiting one or more additional symptoms of malaria, including those of severe malaria (e.g., severe malarial anemia, respiratory distress, shock, spontaneous bleeding, hypoglycemia, repeated seizures, hemoglobinuria, hypoglycemia, prostration, impaired consciousness, jaundice, hyperparasitemia). Patients include adults and children (e.g., ages 5-12 years).

### 5.5. Pharmaceutical Formulations

Pharmaceutical compositions include single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed. (Mack Publishing, Easton Pa.: 1990).

### 5.5.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See, e.g., Remington's Pharmaceutical Sciences, 18^{th} ed. (Mack Publishing, Easton Pa.: 1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. Liquid oral dosage forms are preferred for most patients suffering from CM.

### 5.5.2. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

Aspects of this invention can be understood from the following examples, which do not limit its scope.

### 6.1. Synthesis of (E/Z)-1-(4-((1R,2S,3R)-1,2,3,4-tetrahydroxybutyl)-1H-imidazol-2-yl)-ethanone oxime

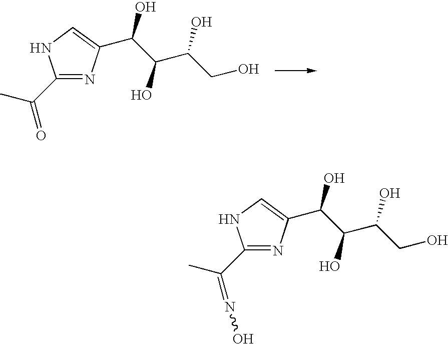

1-[4-((1R,2S,3R)-1,2,3,4-Tetrahydroxy-butyl)-1H-imidazol-2-yl]-ethanone (THI, prepared according to Halweg, K. M. and Büchi, G., J. Org. Chem. 50:1134-1136 (1985)) (350 mg, 1.52 mmol) was suspended in water (10 ml). Hydroxylamine hydrochloride (126.8 mg, 1.82 mmol, 1.2 eq.) and sodium acetate (247.3 mg, 3.04 mmol. 2 eq.) was added, and the suspension was stirred at 50° C. The reaction mixture turned clear after approximately 4 hours. Stirring was continued at 50° C. for 16 hours. LCMS analysis indicated the formation of the product and the absence of starting material. The reaction mixture was allowed to attain room temperature and passed through a fine porosity filter. This solution was used directly to purify the product by using preparative HPLC: Atlantis HILIC silica column 30×100 mm; 2%-21% water in acetonitrile over 6 minutes; 45 ml/min; with detection at 254 nm. The product fractions were collected and the acetonitrile was evaporated under reduced pressure. The aqueous solution was lyophilized to yield the product, a mixture of approximately 3:1 anti:syn isomers, as a white solid: 284 mg (77%).

LCMS: Sunfire C-18 column, 4.6×50 mm; 0-17% MeOH (0.1% TFA) in water (0.1% TFA) over 5 min; flow rate=3 ml/min; Detection 220 nm; Retention times: 0.56 min (syn isomer, 246.0 (M+1)) and 0.69 min (anti isomer, 246.0 (M+1)). ¹H NMR (D₂O and DCl) δ 2.15 and 2.22 (singlets, 3H), 3.5-3.72 (m, 4H), 4.76 (br, OH protons and H₂O), 4.95 and 4.97 (singlets, 1H), 7.17 and 7.25 (singlets, 1H). ¹³C NMR (D₂O and DCl) δ 10.80, 16.76, 63.06, 64.59, 64.75, 70.86, 72.75, 72.85, 117.22, 117.64, 135.32, 138.39, 141.35, 144.12.

### 6.2. Synthesis of (E)-1-(4-((1R,2S,3R)-1,2,3,4-tetrahydroxybutyl)-1H-imidazol-2-yl)-ethanone oxime

This compound was prepared in two steps, as shown below

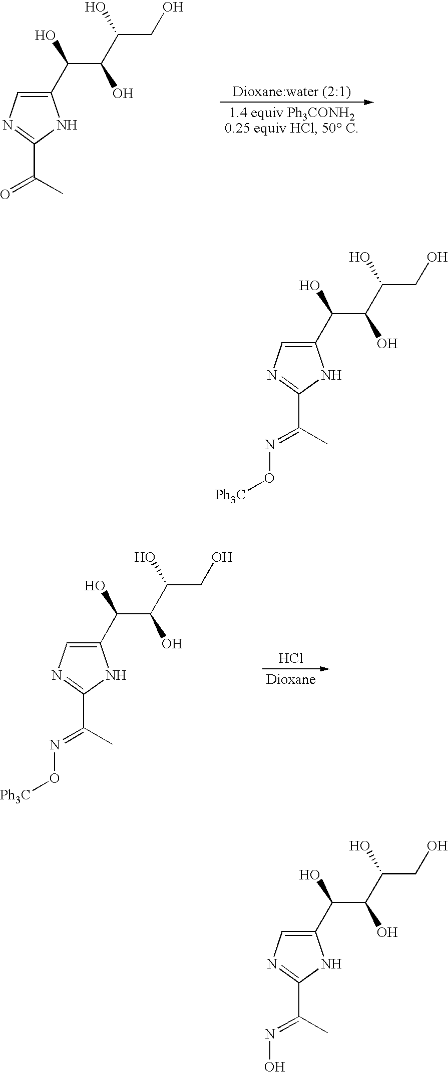

First, to a flask charged with THI (21.20 mmol, 4.88 g) is added water (25 ml) and 1N aqueous HCl (21.2 ml, 21.2 mmol). After all solids dissolved, a solution of trityl hydroxylamine (25.44 mmol, 7.00 g) in dioxane (55 ml) was added and the reaction was maintained at 50° C. for 4 h. At completion, the reaction was cooled room temperature and the solution was adjusted to pH=7 by addition of 1N aqueous NaOH. The neutralized solution was then concentrated to a plastic mass, which was purified by flash chromatography on silica gel [10% MeOH/1% NH₄OH (5% wt. solution in water) in DCM] to provide the trityl-ether as clear plastic. Treatment of the plastic mass with hexane and concentration provided a white foam, which could be dried under vacuum to a flakey solid (10.00 g, 97% yield).

Second, to a vigorously stirred, room temperature solution of the trityl oxime-ether (4.8 g, 10 mmol) in dioxane (90 ml) is added a solution of HCl in dioxane (4M, 60 ml). After a few minutes, a white precipitant was observed, and stirring was continued for a total of 30 minutes, before filtering over a fritted glass filter and rinsing the cake with dioxane and ether. The cake was redissolved in water (200 ml), sonicated for 5 min, then cooled to 0° C., treated with celite (5 g), and filtered over a fitted glass filter. The aqueous solution was concentrated to dryness, then isolated from methanol (30 ml)/diethyl ether (60 ml) to provide the E-oxime as an analytically pure white powder (3.8 g, 80% yield).

### 6.3. Synthesis of (E/Z)-1-(4-((1R,2S,3R)-1,2,3,4-tetrahydroxybutyl)-1H-imidazol-2-yl)ethanone O-methyl oxime

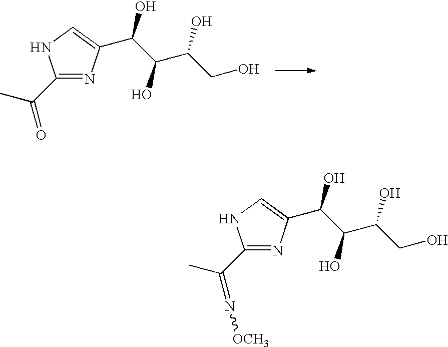

The captioned compound was prepared as described above in Example 6.3, by using methoxylamine hydrochloride in place of hydroxylamine hydrochloride, in 74% yield. The product was a white fluffy solid.

LCMS: Sunfire C-18 column, 4.6×50 mm; 0-17% MeOH (0.1% TFA) in water (0.1% TFA) over 5 min; flow rate=3 ml/min; Detection 220 nm; Retention times: 1.59 minutes (syn isomer, 260.1 (M+1)) and 1.73 min (anti isomer, 260.1 (M+1)). ¹H NMR (D₂O) δ 2.18 and 2.22 (singlets, 3H), 3.54-3.60 (m, 1H), 3.66-3.79 (m, 3H), 3.94 and 3.95 (singlets, 3H), 4.76 (br, OH protons and H₂O), 4.93 and 4.97 (singlets, 1H), 7.17 and 7.25 (singlets, 1H). ¹³C NMR (D₂O) δ 11.55, 17.56, 62.32, 62.38, 62.99, 63.07, 67.09, 71.54, 73.86, 119.09, 138.64, 139.79, 142.95, 144.98, 148.97.

### 6.4. Synthesis of 1-(5-methyl-4-((1R,2S,3R)-1,2,3,4-tetrahydroxybutyl)-1H-imidazol-2-yl)ethanone

The captioned compound was prepared in seven steps, using the process outlined below.

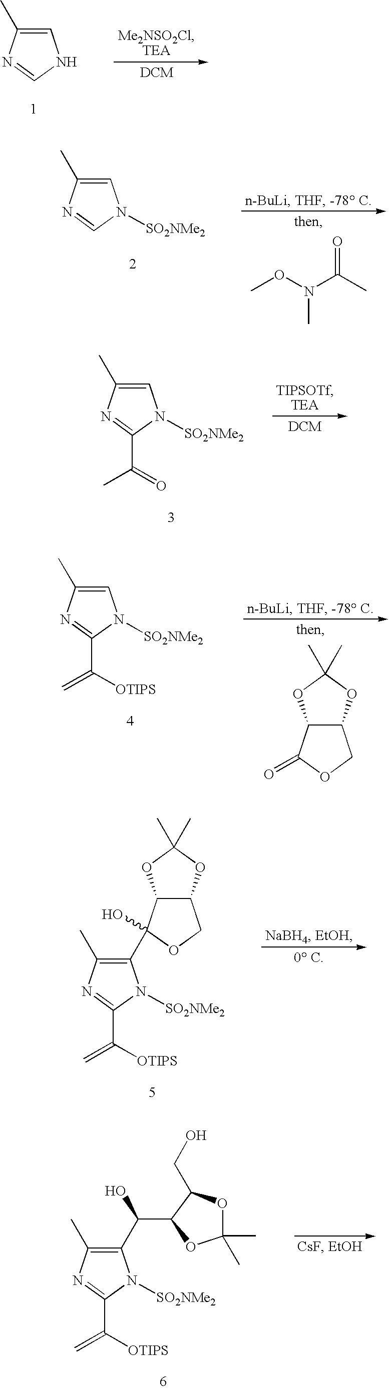

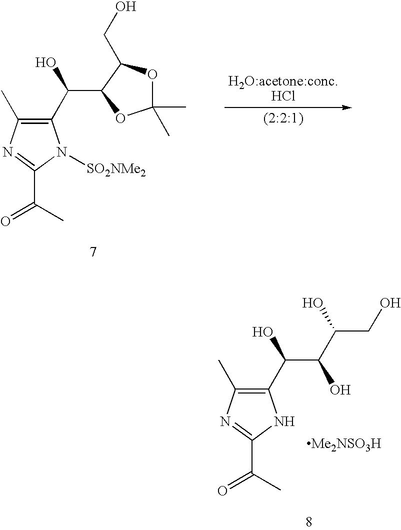

4-Methylimidazole-1-dimethylaminosulfonamide (2): To a room temperature solution of 4-methyl imidazole 1 (3.00 g, 36.54 mmol) in toluene (200 ml) was consecutively added triethylamine 5.6 ml, 40.20 mmol) and N,N-dimethylaminosulfamoyl chloride (3.9 ml, 36.54 mmol). The vessel was stored in a 5° C. refrigerator for 48 hours, then the solids were filtered off from the reaction and the liquor was concentrated to obtain a 2.5:1 mixture of regioisomers 2 and 2a. The crude product was purified by flash chromatography over silica gel (80-100% ethyl acetate:hexane eluent) to obtain a 2:2a in a 5.5:1 mixture of regioisomers (4.31 g, 62% yield): M+1=190.1

4-Methyl-2-acetylimidazole-1-dimethylaminosulfonamide (3): To a −78° C. solution of the imidazole 2 (1.99 g, 10.54 mmol) in tetrahydrofuran (70 ml) was added slowly a solution of n-BuLi in hexane (2.5M, 11.60 ml). After 40 minutes, N-methoxy-N-methylacetamide (1.30 g, 12.65 mmol) was added dropwise to the cooled solution. The reaction was allowed to warm to room temperature and maintained for 2 hours. At completion, the reaction was quenched by addition of saturated aqueous NH₄Cl (20 ml), then diluted with water (20 ml). The layers were separated, and the organic layer was washed with ethyl acetate (2×30 ml). The combined organics were washed with brine (20 ml), then dried over MgSO₄ and concentrated. The crude product was purified by flash chromatography over silica (60-80% ethyl acetate:hexane eluent) to provide 3 as an oil (1.85 g, 76% yield): M+1=232.1.

4-Methyl-2-(1-(triisopropylsilyloxy)vinyl)-1-dimethylaminosulfonamide (4): To a solution of imidazole 3 (1.65 g, 7.14 mmol) in dichloromethane (45 ml) was consecutively added triethylamine (1.00 ml, 14.28 mmol) and triisopropylsilyl trifluoromethanesulfonate (2.12 ml, 7.86 mmol). The reaction was maintained at room temperature for 20 hours, then quenched by the addition of saturated aqueous NaHCO₃ (20 ml). The mixture was diluted with water (20 ml) and the layers were separated. The aqueous layer was washed with dichloromethane (2×20 ml) and the combined organics were washed with brine solution (20 ml), then dried over MgSO₄ and concentrated. The resulting oil was purified by flash chromatography over silica gel (1-2% methanol: dichloromethane eluent) to provide silyl enol ether 4 as an orange oil (2.26 g, 83% yield): M+1=388.2.

Lactol (5): To a −78° C. solution of imidazole 4 (2.26 g, 5.84 mmol) in tetrahydrofuran (40 ml) was slowly added a hexane solution of n-BuLi (2.5M, 3.27 ml). After 30 minutes, a solution of (−)-2,3-O-isopropylidine-D-erythronolactone (1.66 g, 10.51 mmol) in tetrahydrofuran (10 ml) was added slowly to the −78° C. solution. The reaction was maintained at −78° C. for 2 hours, then allowed to warm to 0° C. before quenching the reaction by addition of saturated aqueous NH₄Cl (20 ml). The mixture was diluted with water (10 ml) and the layers were separated. The organics were washed with ethyl acetate (2×20 ml) and the combined organics were washed with brine (20 ml), then dried over MgSO₄ and concentrated. The crude product was purified on silica gel (30-50% ethyl acetate:hexane eluent) to provide the lactol 5 (2.69 g, 85% yield) as a white foam: M+1=546.4.

Diol (6): To a 0° C. solution of the lactol 5 (2.09 g, 3.83 mmol) in ethanol (70 ml) was added granular NaBH₄ (1.4 g, 38.32 mmol) in a few portions. After 2 hours, the reaction was warmed to room temperature for 30 minutes, then concentrated. The residue was redissolved in water (40 ml) and ethyl acetate (40 ml). The biphasic mixture was stirred vigorously for 10 minutes, then the layers were separated. The aqueous layer was washed with ethyl acetate (2×40 ml) and the combined organics were washed with brine (30 ml), then dried over MgSO₄ and concentrated. The crude foam was purified by flash chromatography over silica (5% methanol:dichloromethane eluent) to provide diol 6 (1.88 g, 90% yield) as a 3:1 mixture of inseparable diasteromers at the benzylic position: M+1=547.4.

Imidazole (7): Cesium fluoride (315 mg, 2.08 mmol) was added to a solution of the imidazole 6 (567 mg, 1.04 mmol) in ethanol (10 ml) and warmed to 65° C. After 1 hour, the reaction was cooled to room temperature and treated with saturated aqueous NH₄Cl (1 ml), then concentrated. The crude product was purified by flash chromatography over silica gel (5% methanol:dichloromethane eluent) to provide imidazole 7 (380 mg, 94% yield) as a white foam: M+1=392.1.

Final Product (8): The protected imidazole 7 (380 mg, 0.97 mmol) was dissolved in acetone (6 ml) and consecutively treated with water (6 ml) and concentrated aqueous HCl (3 ml). The vessel was warmed to 40° C. for 45 minutes, then cooled to room temperature and concentrated. The crude material was purified by reverse phase preparative chromatography using a 150 mm×30 mm Zorbax C-6 column using unbuffered solvents by the following method: 1% acetonitrile:water isocratic run for 5 minutes (T_{R}=1.52 minutes). Following lyophylization, compound 8 was obtained as the dimethylaminosulfamic acid salt an amorphous solid: M+1=245.1; ¹H NMR (400 MHz, CDCl₃) major δ 5.04 (d, 1H), 3.62 (comp. m, 2H), 3.42 (comp. m, 2H), 2.62 (s, 6H), 2.43 (s, 3H), 2.21 (s, 3H); minor δ 5.01 (d, 1H), 3.79 (comp. m, 2H), 3.55 (comp. m, 2H), 2.62 (s, 6H), 2.43 (s, 3H), 2.21 (s, 3H).

### 6.5. Synthesis of (1R,2S,3R)-1-(2-(1-hydrazonoethyl)-1H-imidazol-4-yl)butane-1,2,3,4-tetraol

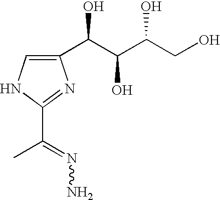

1-[4-((1R,2S,3R)-1,2,3,4-Tetrahydroxy-butyl)-1H-imidazol-2-yl]-ethanone (THI, prepared according to Halweg, K. M. and Büchi, G., J. Org. Chem. 50:1134-1136 (1985)) (148 mg, 0.64 mmol) was suspended in methanol (3 ml) and water (1 ml). Hydrazine hydrate (35 mg, 0.7 mmol, 1.2 eq.) and acetic acid (one drop) were added, and the suspension was stirred at 50° C. for 6 hours. LCMS analysis indicated the formation of the product and the absence of starting material. The reaction mixture was cooled to room temperature and diluted with tetrahydrofuran. The resulting white precipitate was collected and washed with tetrahydrofuran to yield the product, a mixture of approximately 3:1 E:Z isomers, as a white solid: 90 mg (58%).

LCMS: Zorbax C-8 column, 4.6×150 mm; 10-90% in water (10 mM ammonium acetate) over 6 min; flow rate=2 ml/min; Detection 220 nm; Retention times: 0.576 min (syn isomer, 245.0 (M+1)) and 1.08 min (anti isomer, 245.0 (M+1)). ¹H NMR (DMSO-d6) δ 2.5 (singlet, 3H under DMSO), 3.4-3.7 (m, 4H), 4.3 (m, 2H), 4.6 (m, 2H), 4.8 (m, 1H), 4.9 and 5.0 (doublets, 1H), 7.04 and 7.21 (singlets, 1H).

### 6.6. Synthesis of N′-(1-(4-((1R,2S,3R)-1,2,3,4-tetrahydroxybutyl)-1H-imidazol-2-yl)ethylidene)acetohydrazide

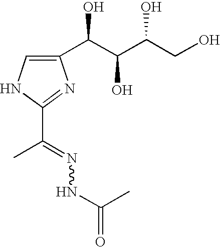

1-[4-((1R,2S,3R)-1,2,3,4-Tetrahydroxy-butyl)-1H-imidazol-2-yl]-ethanone (160 mg, 0.70 mmol) was suspended in methanol (3 ml) and water (1 ml). Acetic hydrazide (56 mg, 0.75 mmol, 1.2 eq.) and hydrochloric acid (one drop, 12 N) were added, and the suspension was stirred at 50° C. for 48 hours. LCMS analysis indicated the formation of the product and the absence of starting material. The reaction mixture was cooled to room temperature and diluted with tetrahydrofuran. The resulting white precipitate was collected and washed with tetrahydrofuran to yield the product, a mixture of approximately 3:1 E:Z isomers, as a white solid: 129 mg (65%).

LCMS: Sunfire C-18 column, 4.6×50 mm; 2-20% in water (10 mM ammonium acetate) over 2.5 min; flow rate=3.5 ml/min; Detection 220 nm; Retention time: 0.53 min (287.1 (M+1)). ¹H NMR (DMSO-d6) δ 2.2 (singlets, 3H), 2.5 (singlets, 3H under DMSO), 3.4-3.7 (m, 4H), 4.3 (br, 2H), 4.6-5.0 (br, 4H), 7.0 (br, 1H), 10.30 and 10.37 (singlets, 1H).

### 6.7. Synthesis of (E)-4-methyl-N′-(1-(4-((1R,2S,3R)-1,2,3,4-tetrahydroxybutyl)-1H-imidazol-2-yl)ethylidene)benzenesulfonohydrazide

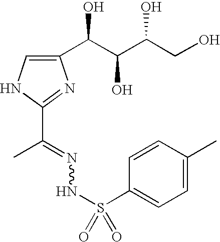

1-[4-((1R,2S,3R)-1,2,3,4-Tetrahydroxy-butyl)-1H-imidazol-2-yl]-ethanone (153 mg, 0.67 mmol) was suspended in methanol (3 ml) and water (1 ml). P-toluenesulfonyl hydrazide (140 mg, 0.75 mmol, 1.2 eq.) and hydrochloric acid (one drop, 12 N) were added, and the suspension was stirred at 50° C. for 24 hours. LCMS analysis indicated the formation of the product and the absence of starting material. The reaction mixture was cooled to room temperature and dry-loaded on silica gel. Flash chromatography on silica gel (10 g SiO₂, 4:1 ethyl acetate:methanol) to yield the product, a mixture of approximately 85:15 E:Z isomers, as a white solid: 142 mg (53%).

LCMS: Sunfire C-18 column, 4.6×50 mm; 10-90% in water (10 mM ammonium acetate) over 2.5 min; flow rate=3.5 ml/min; Detection 220 nm; Retention times: 0.50 min (399.2 (M+1)) and 0.66 min (399.3 (M+1)). ¹H NMR (Methanol-d4) δ 2.2 (singlets, 3H), 2.41 and 2.45 (singlets, 3H), 3.6-3.85 (m, 4H), 4.99 and 5.05 (singlets, 1H), 7.09 (br s, 1H), 7.39 (d, 2H, j=8 Hz), 7.77 and 7.87 (d, 2H, j=8 Hz).

### 6.8. Synthesis of N′-(1-(4-((1R,2S,3R)-1,2,3,4-tetrahydroxybutyl)-1H-imidazol-2-yl)ethylidene)benzohydrazide

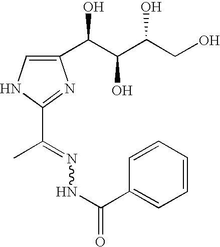

1-[4-((1R,2S,3R)-1,2,3,4-Tetrahydroxy-butyl)-1H-imidazol-2-yl]-ethanone (150 mg, 0.65 mmol) was suspended in methanol (3 ml) and water (1 ml). Benzoic acid hydrazide (102 mg, 0.75 mmol, 1.2 eq.) and hydrochloric acid (one drop, 12 N) were added, and the suspension was stirred at 50° C. for 18 hours. LCMS analysis indicated the formation of the product and the absence of starting material. The homogeneous reaction mixture was cooled to room temperature and concentrated in vacuo. C-18 Reverse-Phase SPE (10 g Alltech Hi-load C18, gradient from water to 20% methanol/water) to yield the product, a mixture of approximately 1:1 E:Z isomers, as a colorless solid: 193 mg (85%).

LCMS: Sunfire C-18 column, 4.6×50 mm; 10-90% in water (10 mM ammonium acetate) over 2.5 min; flow rate=3.5 ml/min; Detection 220 nm; Retention time: 0.49 min (349.2 (M+1)). ¹H NMR (Methanol-d4) δ 2.2 (singlets, 3H), 2.42 and 2.45 (singlets, 3H), 3.6-3.85 (m, 4H), 5.11 and 5.14 (singlets, 1H), 7.30 (br s, 1H), 7.40-7.7 (m, 4H), 7.80 and 7.95 (m, 2H), 8.1 (br s, 1H).

### 6.9. Synthesis of (E)-ethyl 2-(1-(4-((1R,2S,3R)-1,2,3,4-tetrahydroxybutyl)-1H-imidazol-2-yl)ethylidene)hydrazinecarboxylate

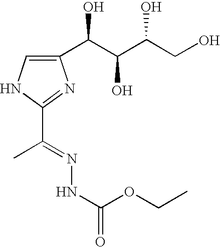

1-[4-((1R,2S,3R)-1,2,3,4-Tetrahydroxy-butyl)-1H-imidazol-2-yl]-ethanone (150 mg, 0.65 mmol) was suspended in methanol (3 ml) and water (1 ml). Ethyl carbazate (78 mg, 0.75 mmol, 1.2 eq.) and hydrochloric acid (one drop, 12 N) were added, and the suspension was stirred at 50° C. for 18 hours. LCMS analysis indicated the formation of the product and the absence of starting material. The reaction mixture was cooled to room temperature, concentrated in vacuo, and diluted with acetone. The resulting white precipitate was collected and washed with acetone to yield the product, one apparent isomer, as a white solid: 96 mg (47%).

LCMS: Sunfire C-18 column, 4.6×50 mm; 2-20% in water (10 mM ammonium acetate) over 2.5 min; flow rate=3.5 ml/min; Detection 220 nm; Retention time: 0.25 min (317.35 (M+1)). ¹H NMR (Methanol-d4) δ 1.36 (t, 3H, j=8 Hz), 2.28 (s, 3H), 2.42 and 2.45 (singlets, 3H), 3.60-3.85 (m, 4H), 4.34 (dd, 2H, j=8 Hz), 5.08 (s, 1H), 7.27 (s, 1H).

### 6.10. Synthesis of (E)-N′-(1-(4-((1R,2S,3R)-1,2,3,4-tetrahydroxybutyl)-1H-imidazol-2-yl)ethylidene)nicotinohydrazide

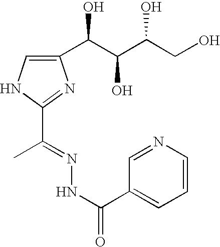

1-[4-((1R,2S,3R)-1,2,3,4-Tetrahydroxy-butyl)-1H-imidazol-2-yl]-ethanone (215 mg, 0.93 mmol) was suspended in methanol (3 ml) and water (1 ml). Nicotinic acid hydrazide (137 mg, 1.0 mmol, 1.1 eq.) and hydrochloric acid (one drop, 12 N) were added, and the suspension was stirred at 50° C. for 48 hours. LCMS analysis indicated the formation of the product and the absence of starting material. The reaction mixture was cooled to room temperature, and partially concentrated in vacuo. The resulting white precipitate was collected and washed with water to yield the product, one apparent isomer, as a white solid: 311 mg (95%).

LCMS: Sunfire C-18 column, 4.6×50 mm; 10-90% in water (10 mM ammonium acetate) over 2.5 min; flow rate=3.5 ml/min; Detection 220 nm; Retention time: 0.22 min (350.27 (M+1)). ¹H NMR (DMSO-d₆) δ 2.37 (s, 3H), 3.60-3.85 (m, 4H), 4.40 (m, 2H), 4.71 (m, 1H), 5.01 (m, 2H), 5.16 (m, 1H), 7.25 (br, 1H), 7.64 (br, 1H). 8.35 (br, 1H). 8.80 (br, 1H). 9.14 (br, 1H).

### 6.11. Synthesis of 3-chloro-N′-(1-(4-((1R,2S,3R)-1,2,3,4-tetrahydroxybutyl)-1H-imidazol-2-yl)ethylidene)benzohydrazide

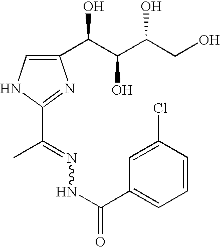

1-[4-((1R,2S,3R)-1,2,3,4-Tetrahydroxy-butyl)-1H-imidazol-2-yl]-ethanone (194 mg, 0.84 mmol) was suspended in ethanol (4 ml) and water (1 ml). 3-chlorobenzoic acid hydrazide (170 mg, 1.0 mmol, 1.2 eq.) and hydrochloric acid (one drop, 12 N) were added, and the suspension was stirred at 50° C. for 48 hours. LCMS analysis indicated the formation of the product and the absence of starting material. The reaction mixture was cooled to room temperature, and partially concentrated in vacuo. The resulting white precipitate was collected and washed with ethanol to yield the product, as a −3:1 E:Z mixture, as a white solid: 108 mg (33%).

LCMS: Sunfire C-18 column, 4.6×50 mm; 10-90% in water (10 mM ammonium acetate) over 2.5 min; flow rate=3.5 ml/min; Detection 220 nm; Retention time: 0.63 min (383.23 (M+1)). ¹H NMR (Methanol-d4) δ 2.44 (s, 3H), 3.60-3.90 (m, 4H), 5.12 (s, 1H), 7.29 (s, 1H), 7.65 (m, 2H), 8.04 (m, 2H).

### 6.12. Synthesis of (E)-4-fluoro-N′-(1-(4-((1R,2S,3R)-1,2,3,4-tetrahydroxybutyl)-1H-imidazol-2-yl)ethylidene)benzohydrazide

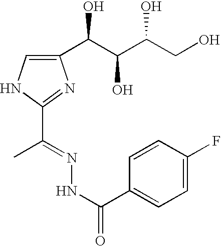

1-[4-((1R,2S,3R)-1,2,3,4-Tetrahydroxy-butyl)-1H-imidazol-2-yl]-ethanone (172 mg, 0.74 mmol) was suspended in ethanol (4 ml) and water (1 ml). 4-fluorobenzoic acid hydrazide (131 mg, 0.85 mmol, 1.1 eq.) and hydrochloric acid (one drop, 12 N) were added, and the suspension was stirred at 55° C. for 48 hours. LCMS analysis indicated the formation of the product and the absence of starting material. The reaction mixture was cooled to room temperature, and partially concentrated in vacuo. The resulting white precipitate was collected and washed with ethanol to yield the product, as one apparent isomer, as a white solid: 97 mg (35%).

LCMS: Sunfire C-18 column, 4.6×50 mm; 10-90% in water (10 mM ammonium acetate) over 2.5 min; flow rate=3.5 ml/min; Detection 220 nm; Retention time: 0.55 min (367.24 (M+1)). ¹H NMR (Methanol-d4, one drop DCl) δ 2.55 (s, 3H), 3.60-3.90 (m, 4H), 5.22 (s, 1H), 7.30 (m, 2H), 7.54 (s, 1H), 8.08 (m, 2H).

### 6.13. Synthesis of (E)-6-amino-N′-(1-(4-((1R,2S,3R)-1,2,3,4-tetrahydroxybutyl)-1H-imidazol-2-yl)ethylidene)nicotinohydrazide

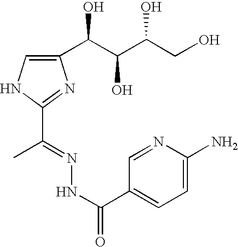

1-[4-((1R,2S,3R)-1,2,3,4-Tetrahydroxy-butyl)-1H-imidazol-2-yl]-ethanone (115 mg, 0.50 mmol) was suspended in ethanol (4 ml) and water (1 ml). Substituted hydrazide (91 mg, 0.6 mmol, 1.2 eq.) and hydrochloric acid (one drop, 12 N) were added, and the suspension was stirred at 55° C. for 48 hours. LCMS analysis indicated the formation of the product and the absence of starting material. The reaction mixture was cooled to room temperature, and partially concentrated in vacuo. The resulting white precipitate was collected and washed with ethanol to yield the product, as one apparent isomer, as a white solid: 136 mg (75%).

LCMS: Sunfire C-18 column, 4.6×50 mm; 10-90% in water (10 mM ammonium acetate) over 2.5 min; flow rate=3.5 ml/min; Detection 220 nm; Retention time: 0.15 min (365.32 (M+1)). ¹H NMR (Methanol-d4, one drop DCl) δ 2.58 (s, 3H), 3.60-3.90 (m, 4H), 5.22 (s, 1H), 7.17 (m, 1H), 7.54 (m, 1H), 8.44 (m, 1H), 8.68 (m, 1H).

### 6.14. Synthesis of (E)-N′-(1-(4-((1R,2S,3R)-1,2,3,4-tetrahydroxybutyl)-1H-imidazol-2-yl)ethylidene)isonicotinohydrazide

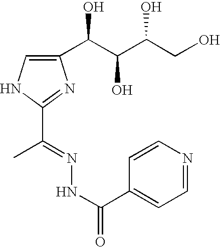

1-[4-((1R,2S,3R)-1,2,3,4-Tetrahydroxy-butyl)-1H-imidazol-2-yl]-ethanone (168 mg, 0.73 mmol) was suspended in ethanol (4 ml) and water (1 ml). Isonicotinic hydrazide (110 mg, 0.80 mmol, 1.1 eq.) and hydrochloric acid (one drop, 12 N) were added, and the suspension was stirred at 55° C. for 24 hours. LCMS analysis indicated the formation of the product and the absence of starting material. The reaction mixture was cooled to room temperature, and partially concentrated in vacuo. The resulting white precipitate was collected and washed with ethanol to yield the product, as one apparent isomer, as a white solid: 136 mg (75%).

LCMS: Sunfire C-18 column, 4.6×50 mm; 10-90% in water (10 mM Ammonium Acetate) over 2.5 min; flow rate=3.5 ml/min; Detection 220 nm; Retention time: 0.15 min (365.32 (M+1)). ¹H NMR (Methanol-d4, one drop DCl) δ 2.63 (s, 3H), 3.60-3.90 (m, 4H), 5.12 (s, 1H), 7.58 (s, 1H), 8.63 (d, 2H, j=8 Hz), 9.14 (d, 2H, j=8 Hz).

### 6.15. Synthesis of (E)-N′-(1-(4-((1R,2S,3R)-1,2,3,4-tetrahydroxybutyl)-1H-imidazol-2-yl)ethylidene)biphenyl-3-carbohydrazide

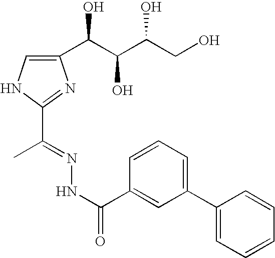

1-[4-((1R,2S,3R)-1,2,3,4-Tetrahydroxy-butyl)-1H-imidazol-2-yl]-ethanone (315 mg, 1.36 mmol) and biphenyl-3-carbohydrazide (360 mg, 1.81 mmol) were suspended in DMSO (2 ml). Concentrated hydrochloric acid (two drops) was added, and the suspension was stirred at 40° C. for 5 hours. LCMS analysis indicated the formation of the product and the absence of starting material. The reaction mixture was cooled to room temperature, diluted with methanol and purified by reverse phase HPLC (10 mM NH₄OAc/acetonitrile). Two fractions (E and Z isomers) of the desired mass were collected separately and lyophized. Fraction one afforded a white solid, 95 mg (16%). Fraction two was a white solid, 82 mg (14%).

Fraction one: Analytical HPLC Zorbax C-8 column, 4.6×150 mm; Solvent A=10 mM ammonium acetate; Solvent B=MeCN; 5% B at 0 min, 5% B at 1 min, 90% B at 3 min, 4 min stop; flow rate=3 ml/min; Detection 220 nm; Retention time: 2.9 min (note: contains ˜5% of the other isomer). M+H=425.28. ¹H NMR (DMSO-d6 with 2 drops D₂O) δ 2.3 (singlet, 3H), 3.3-3.7 (m, 4H), 4.9 (m, 1H), 7.19 (s, 1H), 7.37 (m, 1H) 7.47 (m, 2H), 7.67 (m, 3H), 7.85-7.92 (m, 2H) and 8.15 (s, 1H). HSQC of the same sample correlated the proton signal at 2.3 (CH₃) with a carbon signal at 20 ppm.

Fraction two: Analytical HPLC Zorbax C-8 column, 4.6×150 mm; Solvent A=10 mM ammonium acetate; Solvent B=MeCN; 5% B at 0 min, 5% B at 1 min, 90% B at 3 min, 4 min stop; flow rate=3 ml/min; Detection 220 nm; Retention time: 2.963 min (note: contains ˜6% of the other isomer). M+H=425.28. ¹H NMR (DMSO-d₆ with 2 drops D₂O) δ 2.4 (singlet, 3H), 3.4-3.6 (m, 4H), 4.77 and 4.86 (broad singlets, combined=1H), 6.9 and 7.1 (broad singlets, combined=1H), 7.40 (m, 1H) 7.50 (m, 2H), 7.61 (m, 1H), 7.73 (m, 2H), 7.87 (m, 2H) and 8.10 (s, 1H). HSQC of the same sample correlated the proton signal at 2.4 (CH₃) with a carbon signal at 13 ppm.

### 6.16. Synthesis of N-hydroxy-4-((1R,2S,3R)-1,2,3,4-tetrahydroxybutyl)-1H-imidazole-2-carboxamide

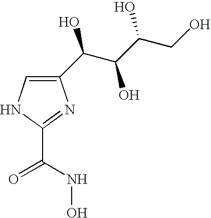

1-[4-((1R,2S,3R)-1,2,3,4-Tetrahydroxy-butyl)-1H-imidazol-2-yl]-ethanone (18 g, 78.3 mmol) was suspended in dichloroethane (160 ml) and 2,2-dimethoxy propane (160 ml). 4-toluenesulfonic acid (3 g) was added and the mixture stirred at 70° C. for 18 hours. The reaction was diluted with dichloromethane and washed with water, 5% bicarbonate, brine and then dry loaded onto SiO₂. Purification by flash chromatography (hexane/ethyl acetate) afforded 1-(4-((4S,4′R,5R)-2,2,2′,2′-tetramethyl-4,4′-bi(1,3-dioxolan)-5-yl)-1H-imidazol-2-yl)ethanone as a colorless oil (18.8 g, 60.6 mmol, 77%; M+H calc: 311.4, obs: 311.3).

The product obtained above (20 g, 64.5 mmol) was dissolved in DMF. K₂CO₃ was added (12.5 g, 90.3 mmol) followed by benzyl bromide (10.7 ml, 90.3 mmol). The reaction was heated at 50° C. for 18 h. LC/MS analysis indicated starting material remained. An additional portion of benzyl bromide (5 ml, 42 mmol) was added and the temperature increased to 60° C. After 3 hours the reaction was quenched with cold water and extracted with ethyl acetate. The organic extracts were washed with water, then brine, dried over sodium sulfate, and loaded onto silica gel. Flash chromatography (20 to 40% ethyl acetate in hexane) afforded 1-(1-benzyl-4-((4S,4′R,5R)-2,2,2′,2′-tetramethyl-4,4′-bi(1,3-dioxolan)-5-yl)-1H-imidazol-2-yl)ethanone (16.1 g, 62%).

The intermediate obtained (13 g, 32.5 mmol) was dissolved in dioxane (120 ml) and treated with NaOH (13.2 g) dissolved in commercial bleach (200 ml, 6% NaOCl). After 2 h of vigorous stirring, the reaction was extracted with ethyl acetate. Organic extracts were washed with brine then dried over celite. Filtration and evaporation afforded a solid that was further dried in vacuo to afford 1-benzyl-4-((4S,4′R,5R)-2,2,2′,2′-tetramethyl-4,4′-bi(1,3-dioxolan)-5-yl)-1H-imidazole-2-carboxylic acid (13 g, quantitative yield, M+H calc: 403.2, obs: 403.2).

The product obtained above (600 mg, 1.49 mmol), O-tritylhydroxylamine (820 mg, 2.98 mmol), EDAC (430 mg, 2.24 mmol) and HOBt (305 mg, 2.24 mmol) were combined with DMF (8 ml) and triethylamine (622 μl, 4.47 mmol). The reaction was stirred at ambient temperature for 22 h, concentrated and then loaded onto silica using DCM/MeOH. Flash chromatography (MeOH/DCM) afforded 1-benzyl-4-((4S,4′R,5R)-2,2,2′,2′-tetramethyl-4,4′-bi(1,3-dioxolan)-5-yl)-N-(trityloxy)-1H-imidazole-2-carboxamide (480 mg, 0.73 mmol, 49%, M+H calc: 660.3, obs: 660.4).

The product obtained above (480 mg, 0.73 mmol) was dissolved in ethanol (50 ml). Pd(OH)₂ (500 mg, 20% on carbon, wet) was added and the reaction stirred under H₂ (65 psi) for 18 h and filtered. Ethanol was removed in vacuo. The residue was dissolved in DCM and purified by flash chromatography (MeOH/DCM) to afford N-hydroxy-4-((4S,4′R,5R)-2,2,2′,2′-tetramethyl-4,4′-bi(1,3-dioxolan)-5-yl)-1H-imidazole-2-carboxamide (150 mg, 0.46 mmol, 63%, M+H calc: 328.1, obs: 328.3).

The product obtained above (150 mg, 0.46 mmol) was dissolved in acetone (8 ml) and water (8 ml). The reaction was cooled to an internal temperature −15° C. using a dry ice/acetone bath. Concentrated HCl (3 ml) was added at a rate such that the internal temperature remained below −10° C. The cold bath was removed and the reaction stirred at ambient temperature for 3 hours, at 4° C. for 18 h and again at ambient temperature for 7 hours. After removal of the acetone and some water in vacuo, a precipitate formed. Dioxane (20 ml) was added followed by THF (10 ml). The solid was isolated by filtration, washed with THF/dioxane and dried in vacuo to afford N-hydroxy-4-((1R,2S,3R)-1,2,3,4-tetrahydroxybutyl)-1H-imidazole-2-carboxamide as the hydrochloride salt (98 mg, 0.40 mmol, 87%).

Mass spec.: M+H calc: 248.1, obs: 248.2. Analytical HPLC: Luna Pheny-Hexyl, 5 um, 4.6×50 mm, isocratic 10 mM ammonium acetate with 1% acetonitrile, flow rate=3 ml/min, 220 nm detection, retention time=0.245 min. ¹H NMR (DMSO-d6) δ 3.37-3.64 (m, 4H), 4.96 (broad singlet, 1H), 7.47 (s, 1H), 11.9 (broad singlet, 1H).

### 6.17. Synthesis of (1R,2S,3R)-1-(2-(5-methylisoxazol-3-yl)-1H-imidazol-5-yl)butane-1,2,3,4-tetraol

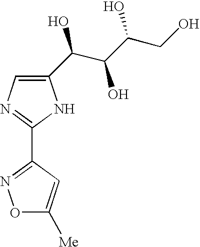

The captioned compound was prepared by General Method A, which is shown below in Scheme 2:

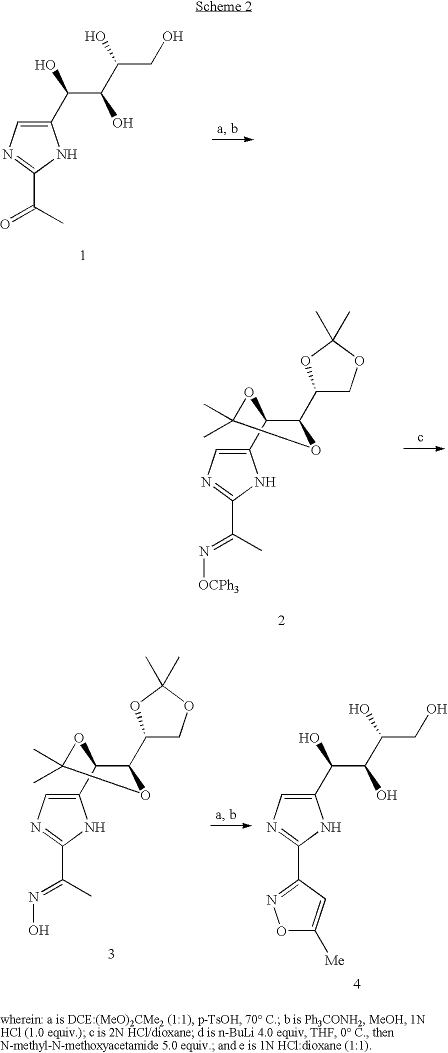

In particular, to a slurry of 1 (4.34 g, 18.87 mmol) in dichloromethane (30 ml) was added 2,2-dimethoxypropane (30 ml) followed by p-toluenesulfonic acid monohydrate (900 mgs, 4.72 mmol). The slurry was heated to 70° C. for 16 h, then cooled to room temperature, and treated with excess triethylamine (1 ml). The reaction was concentrated and dried by toluene azeotrope to give an amber solid that was carried on immediately without purification.

The amber solid was dissolved in MeOH (100 ml), and then treated with N-trityl hydroxylamine (6.75 g, 24.53 mmol) and 1N HCl (18.5 ml, 18.5 mmol). The reaction became clear after 1 h, and was maintained at room temperature for 18 h. At completion, the reaction was neutralized to pH=7 with 10N NaOH solution, then concentrated under reduced pressure. The crude material was purified by chromatography on silica gel (32-63 μm, 10% MeOH:CH₂Cl₂ w/1% NH₄OH) to provide the protected product 2 (9.8 g, 91% yield, 2 steps) as a white foam.

Anhydrous 4M dioxane (20 ml) was added to a solution of 2 (3.11 g, 5.48 mmol) in anhydrous dioxane (40 ml). After 1 h, the reaction was concentrated under vacuum, then redissolved in anhydrous DCM (60 ml), treated with excess triethylamine (5 ml), then concentrated again. The crude product was flashed over silica gel (3-8% MeOH:CH₂Cl₂ w/0.5-1.0% NH₄OH) to provide the oxime 3 (1.05 g, 59% yield) as a white foam.

To a −45° C. solution of 3 (500 mgs, 1.54 mmol) in THF (15 ml) was added dropwise a 1.6 M hexane solution of n-BuLi (3.85 ml, 6.16 mmol). After 10 min, N-methyl-N-methoxyacetamide (0.79 ml, 7.69 mmol) was added dropwise and the reaction was allowed to warm to room temperature. After 2 h, the reaction was quenched by addition of NH₄Cl (10 ml) and diluted with water (5 ml) to dissolve solids. The layers were separated and the aqueous layer was extracted with Et₂O (2×20 ml). The combined organics were washed with brine (25 ml), then dried over MgSO₄ and concentrated under vacuum. The resulting foam was purified by flash chromatography over silica gel (60-90% EtOAc:hexane) to provide a white foam solid.

To a solution of this intermediate white solid in dioxane (5 ml) was added 1N HCl (5 ml). The reaction was heated to 80° C. for 2 h, and then concentrated under reduced pressure to dryness. The resulting glassy solid was lyophilized from water (8 ml) to provide 4 (224 mgs, 48% yield, 2 steps) as a fluffy white powder. MS m/z C₁₁H₁₅N₃O₅[M+H]⁺=270; ¹H NMR (400 MHz, D₂O): δ 7.54 (s, 1H), 6.7 (s, 1H), 5.2 (s, 1H), 3.83-3.59 (m, 4H), 2.49 (s, 1H); ¹³C NMR (100 MHz, D₂O): δ 174.3, 150.0, 136.6, 135.0, 118.1, 101.0, 73.1, 71.0, 65.0, 63.2.

### 6.18. Synthesis of (1R,2S,3R)-1-(2-(5-ethylisoxazol-3-yl)-1H-imidazol-5-yl)butane-1,2,3,4-tetraol

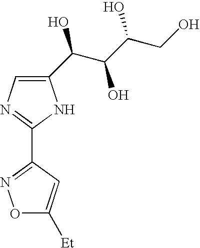

This compound was synthesized by General Method A, by alkylating intermediate 3 with N-methyl-N-methoxy ethyl amide. MS m/z C₁₂H₁₇N₃O₅[M+H]⁺=284; ¹H NMR (400 MHz, D₂O): δ 7.24 (s, 1H), 6.54 (s, 1H), 4.95 (s, 1H), 3.84-3.56 (m, 4H), 2.82-2.77 (m, 2H), 1.25 (t, J=6.0 Hz, 3H).

### 6.19. Synthesis of (1R,2S,3R)-1-(2-(isoxazol-3-yl)-1H-imidazol-5-yl)butane-1,2,3,4-tetraol

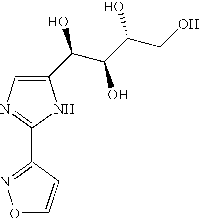

This compound was prepared by modifying General Method A as shown below in Scheme 3:

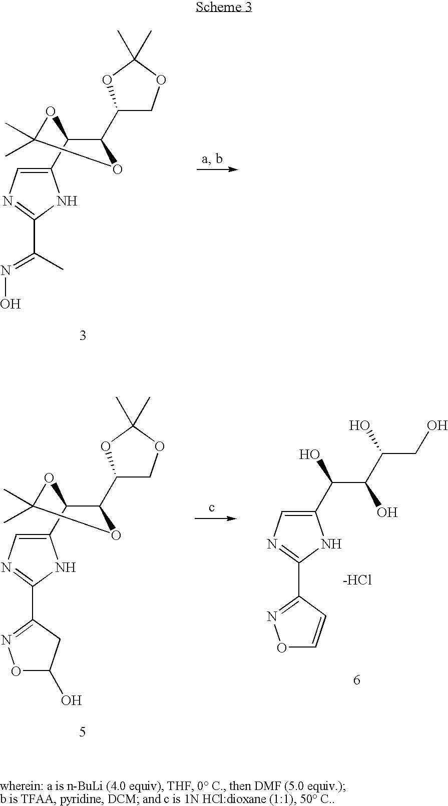

In particular, to a −45° C. solution of 3 (424 mgs, 1.30 mmol) in THF (15 ml) was added dropwise a 2.5 M hexane solution of n-BuLi (2.1 ml, 5.25 mmol). After 10 min, anhydrous DMF (0.505 ml, 6.52 mmol) was added dropwise and the reaction was allowed to warm to room temperature. After 2 h, the reaction was quenched by addition of NH₄Cl (10 ml) and diluted with water (5 ml) to dissolve solids. The layers were separated and the aqueous layer was washed with Et₂O (2×20 ml). The combined organics were washed with brine (25 ml), then dried over MgSO₄ and concentrated under vacuum. The resulting foam was flashed over silica gel (3-6% MeOH: CH₂Cl₂ with 0.5% NH₄OH) to provide the hemiacetal 5 (220 mgs, 47% yield) as a white foam.

To a 0° C. solution of 5 (130 mgs, 0.37 mmol) in THF was sequentially added pyridine (120 μl, 1.48 mmol) and trifluoroacetic acid anhydride. The reaction was warmed to room temperature for 10 min, and then heated to 55° C. for 16 h. At completion, the reaction was concentrated under vacuum, then purified by flash chromatography over silica gel (60-90% EtOAc:hexane) to provide the heterobicycle bisketal (60 mgs, 47% yield) as a white foam that was finally deprotected using standard acidic conditions to give Example 3 compound as a white crystalline solid. MS m/z C₁₀H₁₃N₃O₅[M+H]⁺=256; ¹H NMR (400 MHz, D₂O) δ 8.87 (s, 1H), 7.55 (s, 1H), 7.05 (s, 1H), 5.21 (s, 1H), 3.75 (m, 3H), 3.63 (m, 2H).

### 6.20. Alternate Synthesis of (1R,2S,3R)-1-(2-(isoxazol-3-yl)-1H-imidazol-5-yl)butane-1,2,3,4-tetraol

The captioned compound was also prepared by the approach referred to herein as General Method B, which is shown below in Scheme 4:

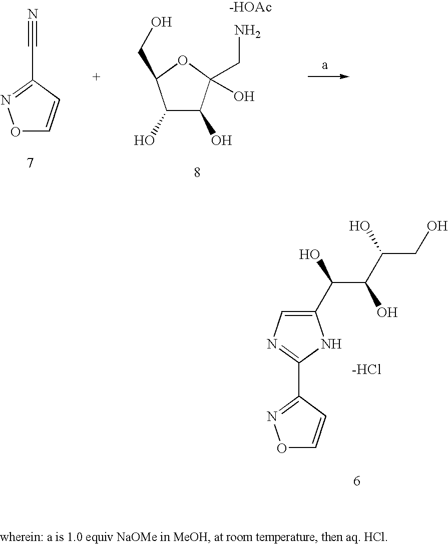

In particular, to a room temperature solution of the nitrile 7 (600 mgs, 6.38 mmol) in MeOH (10 ml) was added 25% w/v MeONa (0.83 ml, 3.83 mmol). After 3 h, fructosamine-acetate (1.53 g, 6.38 mmol) was added and the solution was maintained at room temperature with vigorous stirring for 5 h. Another portion of 25% w/v MeONa (0.66 ml, 3.19 mmol) was then added to the thick slurry. After 16 h, the reaction was filtered and the cake washed with cold MeOH. The cake was then treated with 1N HCl (20 ml) and filtered. The aqueous solution was concentrated under vacuum to constant weight to provide title compound (1.30 g, 66% yield) as a white powder.

### 6.21. Synthesis of (1R,2S,3R)-1-(2-(2-methylthiazol-4-yl)-1H-imidazol-4-yl)butane-1,2,3,4-tetraol

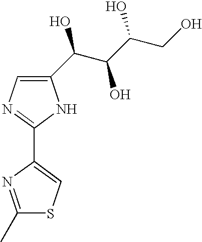

The title compound was prepared by General Method B using 2-methylthiazole-4-carbonitrile (1.023 g, 8.25 mmol), sodium methoxide in methanol (25 wt %, 1.07 ml, 4.95 mmol), methanol (8.25 ml) and compound 8 (2.00 g, 8.26 mmol). After 2.5 days, and additional portion of sodium methoxide in methanol (25 wt %, 0.891 ml, 4.125 mmol) was added. After 24 hours, the solid that had formed was collected by filtration and washed with cold methanol to afford the title compound (1.70 g, 5.96 mmol, 72% yield). MS m/z C₁₁H₁₅N₃O₄S [M+H]=286; ¹H NMR (400 MHz, CD₃OD) δ 2.81 (s, 3H), 3.67-3.75 (m, 2H), 3.77-3.88 (m, 2H), 5.21 (s, 1H), 7.47 (s, 1H), 8.35 (s, 1H).

### 6.22. Synthesis of (1R,2S,3R)-1-(2-(1-benzyl-1H-1,2,4-triazol-3-yl)-1H-imidazol-4-yl)butane-1,2,3,4-tetraol hydrochloride

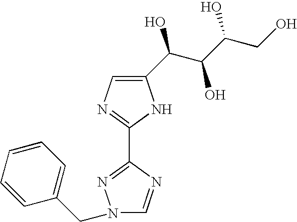

The captioned compound was prepared by General Method B with the following alterations: 1-benzyl-1H-1,2,4-triazole-3-carbonitrile (2.10 g, 11.4 mmol) was dissolved in methanol (12 ml) and treated with sodium methoxide in methanol (25 wt %, 1.48 ml, 6.8 mmol) and stirred for 18 h and 8 was added and the reaction stirred for 18 h. The resulting solid was isolated by filtration, washed with methanol and dried in vacuo to afford a white solid (3.20 g, 9.28 mmol, 81% yield). This solid was suspended in THF (50 ml), cooled in an ice bath and HCl (4 M in dioxane, 7.5 ml, 30 mmol) was added. The ice bath was removed and the suspension was stirred for 4 h. The solid was isolated by filtration, washed with THF and dried in vacuo to afford the title compound (3.50 g, 9.19 mmol, 99% yield) as a shite solid. MS m/z C₁₆H₁₉N₅O₄ [M+H]⁺=346; ¹H NMR (400 MHz, CD₃OD) δ 2.81 (s, 3H), 3.67-3.75 (m, 2H), 3.77-3.88 (m, 2H), 5.21 (s, 1H), 7.47 (s, 1H), 8.35 (s, 1H).

### 6.23. Synthesis of (1R,2S,3R)-1-(1H,1′H-2,2′-biimidazol-5-yl)butane-1,2,3,4-tetraol

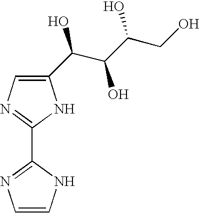

The captioned compound was prepared by General Method B with the following alterations. To a solution of 1H-imidazole-2-carbonitrile (0.39 g, 4.17 mmol) in methanol (4.8 ml) was added a solution of sodium methoxide in methanol (25 wt %, 0.54 g, 0.57 ml, 2.50 mmol), stirred for 16 h and compound 8 (0.964 g, 4.17 mmol) was added in 10 ml of MeOH. A precipitate formed and was filtered and washed with acetone (15 ml). The filtrate was concentrated to dryness, and was purified by preparative HPLC (10 mM aq ammonium acetate/acetonitrile) to give the title compound (0.0141 g, 0.0554 mmol) as an off-white solid. MS m/z C₁₀H₁₄N₄O₄ [M+H]⁺=255; ¹H NMR (400 MHz, CD₃OD) δ 3.56-3.57 (m, 2H), 3.67-3.74 (m, 2H), 4.90 (s, 1H), 7.04 (s, 1H).

### 6.24. Synthesis of (1R,2S,3R)-1-(2-(5-methoxy-4,5-dihydroisoxazol-3-yl)-1H-imidazol-5-yl)butane-1,2,3,4-tetraol

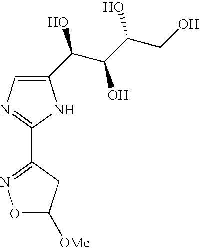

A 1M solution of HCl (10 ml) was added to a room temperature solution of the imidazole 5 (Scheme 3, 500 mg, 1.41 mmol) in MeOH (10 ml). The reaction was heated to 50° C. for 8 h, cooled to room temperature, and concentrated to dryness to provide the title compound (430 mgs, 100% yield) as a slightly yellow powder as a 1:1 mixture of diastereomers. MS m/z C₁₁H₁₇N₃O₆ [M+H]⁺=288; ¹H NMR (400 MHz, D₂O) δ 7.06 (s, 1H), 5.71 (d, J=7.2 Hz) and 5.41 (d, J=7.2 Hz, 1H), 4.72 (s, 1H), 3.2-3.4 (m, 3H), 2.98-2.80 (m, 2H).

### 6.25. Synthesis of (1R,2S,3R)-1-(2-(5-methyl-1H-pyrazol-3-yl)-1H-imidazol-5-yl)butane-1,2,3,4-tetraol

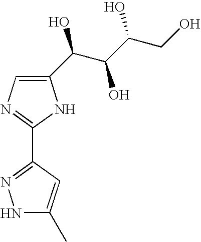

The title compound was prepared from 1-(5-((4S,4′R,5R)-2,2,2′,2′-tetramethyl-4,4′-bi(1,3-dioxolan)-5-yl)-1H-imidazol-2-yl)ethanone (compound 9) as follows. A solution of 9 (975 mg, 3.15 mmol) in THF (15 ml) was added slowly to a −10° C. solution of potassium hexamethyldisilazane (15.72 ml of a 0.5 M toluene solution, 7.86 mmol) in THF (15 ml). The reaction was maintained at −10° C. for 10 min before the addition of ethyl acetate (1.55 ml, 15.75 mmol). The reaction was warmed to room temperature for 1 h, then quenched by the addition of 30 ml NH₄Cl (sat. aq.). The layers were separated, and the aqueous layer was washed with EtOAc (2×30 ml). The combined organics were washed with water (30 ml) and brine (30 ml), then dried over MgSO₄ and concentrated. The resulting tan material was used without further purification.

The crude material was dissolved in EtOH (20 ml) and acidified with 1N HCl (5 ml). The stirred, room temperature solution was then treated with excess hydrazine monohydrate (200 μl). At completion, the reaction was adjusted to pH=7 with 1 N NaOH, then concentrated to a ˜10 ml volume. DCM (30 ml) was added to dissolve the solids which had precipitated from the aqueous solution, and the layers were separated. The organic layer was dried over MgSO₄ and concentrated. The crude was flashed over silica (5-10% MeOH:DCM eluent) to provide the protected pyrazole (204 mg, 19% yield) as a clear foam.

A solution of 1N HCl (5 ml) was added to a room temperature solution of the protected heterobicycle (180 mgs, 0.52 mmol), and the reaction was heated to 50° C. After 1.5 h, the reaction was cooled to room temperature, then concentrated to dryness. The foam was re-dissolved in 2 ml MeOH, then triturated with 3 ml Et₂O and cooled to 0° C. before decanting the liquids. The solid was washed with Et₂O (2×2 ml), then dried under a high vacuum to provide the title compound (130 mgs, 70% yield) as a white powder. MS m/z C₁₆H₁₆N₄O₄ [M+H]⁺=269; ¹H NMR (400 MHz, D₂O) δ 7.28 (s, 1H), 6.52 (s, 1H), 5.07 (d, J=0.9 Hz, 1H), 3.74-3.54 (m, 4H), 2.22 (s, 1H); ¹³C NMR (D₂O): δ 142.8, 139.1, 136.3, 134.1, 116.0, 104.0, 72.6, 70.6, 64.4, 62.7, 9.6.

### 6.26. Cerebral Malaria Model

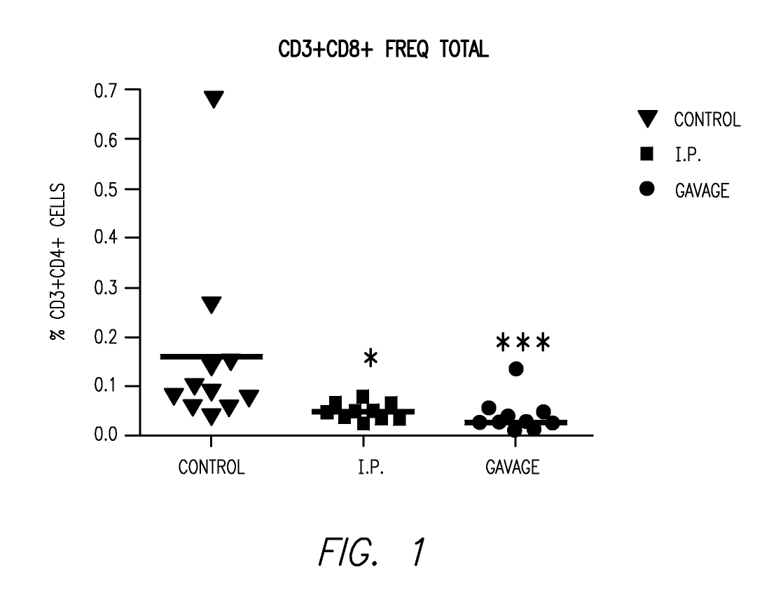

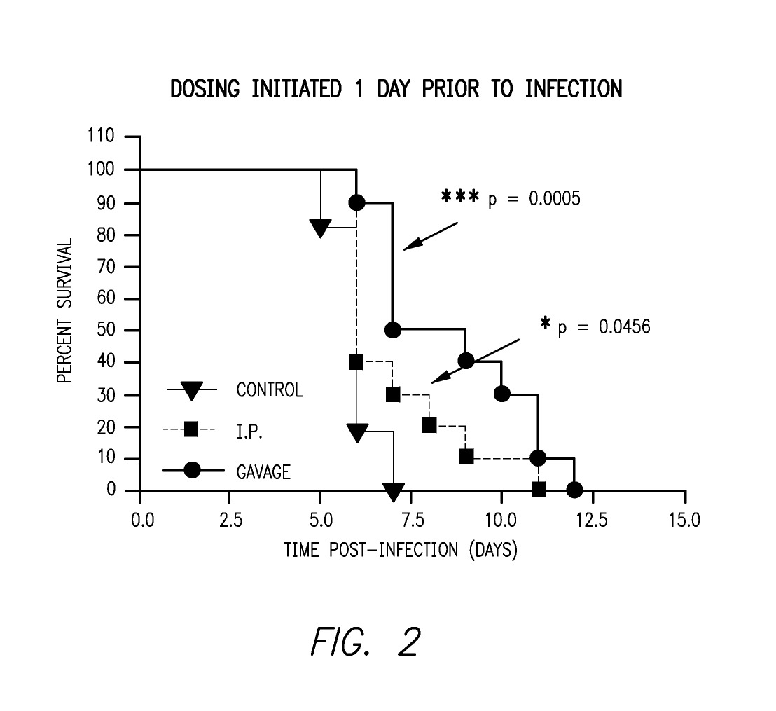

FIGS. 1 and 2 show results of an experiment using three groups of 10 female C56B1/6 mice. The mice in all three groups were infected with 1 million parasites (P. berghei ANKA) i.p. in 500 μl of media. The first group was the control group. The S1P lyase inhibitor (E)-1-(4-((1R,2S,3R)-1,2,3,4-tetrahydroxybutyl)-1H-imidazol-2-yl)-ethanone oxime was administered i.p. (100 mg/kg) to the mice in the second group, and was administered by gavage (100 mg/kg) to the mice in the third group. The drug was administered daily, and was first administered one day before infection.

Twenty four hours after the drug was first administered, tail vein blood was taken from the mice, and flow cytometry analysis was used to assess the levels of B and T cells, using antibodies to CD3, CD4, CD8 and CD19. The animals were monitored daily for body weight, hematocrit, and parasitaemia, and twice daily for survival.

As shown in FIG. 1, mice in both treated groups exhibited decreased CD8+ T cells as compared to those in the untreated, control group. As shown in FIG. 2, the mice in both treated groups lived significantly longer than those in the control group.

All references (e.g., publications, patents, and patent applications) cited herein are incorporated by reference in their entireties.

What is claimed is:

1. A method of treating, managing or preventing cerebral malaria, which comprises administering to a patient in need thereof a therapeutically or prophylactically effective amount of an S1P lyase inhibitor.

2. The method of claim 1, wherein the S1P lyase inhibitor is a compound of the formula: 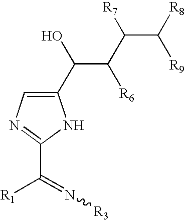
or a pharmaceutically acceptable salt thereof, wherein:
R₁ is hydrogen, alkyl or aryl;
R₃ is OR_{3A}, NHC(O)R_{3A}, NHSO₂R_{3A} or hydrogen;
each R_{3A} is independently hydrogen or alkyl, aryl, alkylaryl, arylalkyl, heteroalkyl, heterocycle, alkylheterocycle, or heterocyclealkyl optionally substituted with halo;
R₆ is OR_{6A} or OC(O)R_{6A};
R₇ is OR_{A} or OC(O)R_{7A};
R₈ is OR_{8A} or OC(O)R_{8A};
R₉ is hydrogen, CH₂OR_{9A} or CH₂OC(O)R_{9A}; and
each R_{6A}, R_{5A}, R_{8A} and R_{9A} is independently hydrogen or lower alkyl.

3. The method of claim 2, wherein the compound is of the formula: 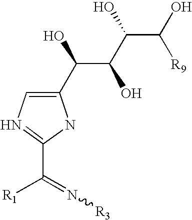
wherein:
R₁ is lower alkyl;
R₃ is hydrogen, OR_{3A}, NHC(O)R_{3A} or NHSO₂R_{3A};
R₉ is hydrogen or CH₂OH; and
each R_{3A} is independently hydrogen or lower alkyl.

4. The method of claim 3, wherein the compound is (E)-1-(4-((1R,2S,3R)-1,2,3,4-tetrahydroxybutyl)-1H-imidazol-2-yl)-ethanone oxime.

5. The method of claim 1, wherein the S1P lyase inhibitor is a compound of the formula: 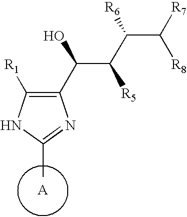
or a pharmaceutically acceptable salt thereof, wherein:
A is an optionally substituted heterocycle;
R₅ is OR_{5A} or OC(O)R_{5A};
R₆ is OR_{6A} or OC(O)R_{6A};
R₇ is OR_{A} or OC(O)R_{7A};
R₈ is hydrogen, CH₂OR_{8A} or CH₂OC(O)R_{8A}; and
each of R_{5A}, R_{6A}, R_{5A} and R_{8A} is independently hydrogen or lower alkyl.

6. The method of claim 5, wherein the compound is of the formula: 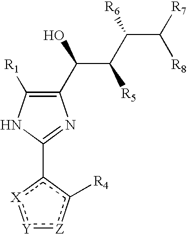
wherein:
X is N or NR₉;
Y is CR₄, N, NR₉, O or S;
Z is CR₄, CHR₄, N, NR₉, O or S;
R₁ is hydrogen or optionally substituted lower alkyl; and
R₃ is optionally substituted alkyl;
each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl; and
each R_{4A} is independently hydrogen or optionally substituted alkyl.

7. The method of claim 5, wherein the compound is of the formula: 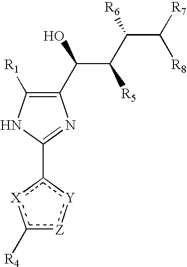
wherein:
X is N or NR₉;
Y is CR₄, N, NR₉, O or S;
Z is CR₄, CHR₄, N, NR₉, O or S;
R₁ is hydrogen or optionally substituted lower alkyl; and
R₃ is optionally substituted alkyl;
each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl; and
each R_{4A} is independently hydrogen or optionally substituted alkyl.

8. The method of claim 1, which further comprises administering to the patient an additional active agent.

9. The method of claim 8, wherein the additional active agent is an anti-malarial drug.

10. The method of claim 9, wherein the anti-malaria drug is quinine, quinidine, artemether or artesunate.

11. The method of claim 8, wherein the additional active agent is an osmotic diuretic (e.g., mannitol, urea).

12. The method of claim 8, wherein the additional active agent is an anti-convulsant (e.g., diazepam, phenyloin, phenobarbital, phenobarbitone).

13. The method of claim 8, wherein the additional active agent is an anti-pyretic (e.g., paracetamol).

14. The method of claim 8, wherein the additional active agent is an anti-oxidant.

15. The method of claim 8, wherein the additional active agent is an anti-inflammatory drug.

16. The method of claim 15, wherein the anti-inflammatory drug is an NSAID, steroid, cyclosporin, thalidomide, revlimid, anti-TNF antibody (e.g., infliximab, etanercept), or pentoxifylline).

17. The method of claim 8, wherein the additional active agent is curdlan sulfate, curcumin, or LMP-420.

18. A pharmaceutical formulation comprising an S1P lyase inhibitor and an additional active agent, wherein the additional active agent is an anti-malarial drug.

19. The formulation of claim 18, wherein the anti-malaria drug is quinine, quinidine, artemether or artesunate.

20. A single unit pharmaceutical dosage form, which comprises an S1P lyase inhibitor and an anti-malarial drug.

21. The dosage form of claim 20, which is suitable for transdermal or topical delivery to a patient.

22. The dosage form of claim 21, which is a patch.
